(19) United States
(12) Patent Application Publication
(10) Pub. No.: US 2009/0197941 A1
(43) Pub. Date: Aug. 6, 2009

(54) Pharmaceutical Compositons for the Treatment of Chronic Obstructive Pulmonary Disease

(76) Inventors: Geoffrey Guy, Salisbury (GB); Philip Robson, Salisbury (GB)

Correspondence Address: WOLF GREENFIELD & SACKS, P.C., 600 ATLANTIC AVENUE, BOSTON, MA, 02210-2206, US

(21) Appl. No.: 12/083,500

(22) PCT Filed: Oct. 11, 2006
(86) PCT No.: PCT/GB2006/003784

§ 371 Date: Mar. 27, 2009

(30) Foreign Application Priority Data
Oct. 12, 2005 (GB) 050751.9

(51) Int. Cl. A61K 31/352; A61P 11/00

(52) U.S. Cl. 514/454

(57) ABSTRACT

The invention relates to the use of a combination of cannabinoids for the treatment of Chronic Obstructive Pulmonary Disease (COPD). Preferably the combination of cannabinoids are cannabidiol (CBD) and delta-9-tetrahydrocannabinol (THC). More preferably the cannabinoids are in a predefined ratio by weight of approximately 1:1 of CBD to THC.

### FIELD OF THE INVENTION

The present invention relates to the use of a combination of cannabinoids for the treatment of Chronic Obstructive Pulmonary Disease (COPD). Preferably the combination of cannabinoids are cannabidiol (CBD) and delta-9-tetrahydrocannabinol (THC). More preferably the cannabinoids are in a predefined ratio by weight of approximately 1:1 of CBD to THC.

### BACKGROUND TO THE INVENTION

Chronic Obstructive Pulmonary Disease (COPD) is a progressive disease of the airways that is characterised by a gradual loss of lung function. The term COPD is often used to describe many different pulmonary conditions including chronic bronchitis, chronic obstructive bronchitis and emphysema. COPD is the fourth highest cause of death in the United States and is projected to be the third leading cause of death for both males and females by 2010.

The symptoms of COPD include a chronic cough, sputum production and a severe disabling shortness of breath. The most important risk factor for COPD is smoking of cigarettes and other types of tobacco, although other causes can be exposure to occupational dusts or chemicals. Outdoor pollution is also thought to be implicated in the cause of COPD as is passive smoking. COPD is usually diagnosed by testing for the presence of an airway obstruction by spirometry and at the present time, as there is no known cure, treatment is generally supportive and is designed to relieve the patients symptoms and to improve their quality of life as much as possible.

It is a factor of the disease that patients that have moderate to severe COPD or those who continue to be exposed to the causative agent such as cigarettes will experience an increasing loss of breath. Acute infections or certain weather conditions may cause a temporary worsening of symptoms (exacerbations) and may result in hospitalisation of the patient.

Exacerbations are often characterised by an increase in the amount of coughing, breathlessness and the production of sputum. It may also be measured by a decrease in the forced expiratory volume measured over one second (FEV₁).

COPD includes, but is not limited to, the diseases chronic bronchitis, (including chronic obstructive bronchitis) and emphysema. These diseases are known to be different to another pulmonary disease: asthma. Asthma is characterised by a muscular spasm in the bronchi and the muscles of the bronchial tree becoming tight causing the lining of the air passages to swell may trigger an asthma attack. This in consequence reduces the amount of airflow and produces the characteristic wheezing heard in asthmatics.

Unlike asthma the obstruction of the airflow in COPD, indicated by an abnormal decline in the FEV₁ is more or less continuous and is largely irreversible.

Whereas in chronic bronchitis the bronchial mucous membrane becomes hypotrophied thereby causing a narrowing of the bronchial lumen. The condition is characterised by an excess of bronchial mucus and a cough that is accompanied by sputum for over 3 months and occurs in at least 2 consecutive years. The symptoms of chronic bronchitis cause an obstruction in the airflow causing respiratory insufficiency and in some cases respiratory failure.

In emphysema damage to the air sacs in the lung (alveoli) means that they unable to completely deflate and in consequence are unable to fill with oxygenated air. The breakdown of the lung architecture can cause breathlessness, chronic cough with or without sputum production and wheezing.

Often patients with moderate to severe COPD remain symptomatic despite maximal medical therapy and as a result often endure a significantly impaired quality of life and emotional well being.

The treatment options for patients with COPD are generally aimed at slowing the progression of the disease, as COPD is not a reversible condition. Medical treatment includes bronchodilators, such beta-2-agonists which relax the smooth muscle thereby decreasing obstruction, anti-inflammatory agents, such as corticosteroids are often used to treat the inflamed airways of COPD sufferers but their long term benefit is unclear.

Other medical treatment options include mucolytics which act to break up the mucus that blocks the airways of patients with COPD. Antibiotics and oxygen therapy are also of use, particularly in patients with frequent exacerbations.

Pulmonary rehabilitation and nutritional support is also used in an attempt to improve the fitness and well being of patients with COPD. Surgical treatment can also be used in severe cases of COPD. Lung volume reduction surgery, where the upper portions of the diseased lungs are removed is a treatment option in only a few patients as is either a single or double lung transplant.

The use of cannabis as a medicine has long been known and during the 19^{th} Century, preparations of cannabis were recommended as a hypnotic sedative which were useful for the treatment of hysteria, delirium, epilepsy, nervous insomnia, migraine, pain and dysmenorrhoea.

Until recent times the administration of cannabis to a patient could only be achieved by preparation of cannabis by decoction in ethanol, which could then be swallowed or by the patient inhaling the vapours of cannabis by smoking the dried plant material. Recent methods have sought to find new ways to deliver cannabinoids to a patient including those which bypass the stomach and the associated first pass effect of the liver which can remove up to 90% of the active ingested dose and avoid the patient having to inhale unhealthy tars and associated carcinogens into their lungs.

Such dosage forms include administering the cannabinoids to the sublingual or buccal mucosae, inhalation of a cannabinoid vapour by vaporisation or nebulisation, enemas or solid dosage forms such as gels, capsules, tablets, pastilles and lozenges.

Cannabinoids, the principle components of cannabis, have a number of pharmacological effects some of which could be of use in the treatment of pulmonary diseases.

Some studies using the cannabinoid tetrahydrocannabinol (THC) have shown that this cannabinoid can be useful in the treatment of asthma. Tashkin et al. (1977) used a nebuliser to administer the THC to the lungs of patients with asthma and concluded that the bronchodilatory effects of the THC were less efficient than those of standard asthma medications.

Williams et al. (1976) conversely showed that aerosolised THC improved ventilatory function. Vachon et al. agreed with this view and stated that micro-aerosolised THC delivered to the lungs was an effective bronchodilator.

The International patent application WO 01/013886 describes the inhalation of THC for lung delivery and the International patent application WO 01/003668 uses liposome encapsulated cannabinoids to be delivered to the pulmonary tissue.

WO 01/58869 describes cannabinoid receptor modulators said to be useful in treating respiratory diseases including chronic pulmonary obstructive disorder and asthma. WO 2004/014825 describes cannabinoid (CB₂) receptor ligands with anti-inflammatory and immunomodulatory activity, said to be useful in treating a range of diseases, including asthma and chronic pulmonary obstructive disorder. These disclosures reflect a prevailing view in the art that compounds capable of modulating cannabinoid receptors may be useful in the treatment of respiratory disease associated with leukocyte activation.

The use of different ratios of cannabinoids such as THC or CBD or their propyl variants, tetrahydrocannabinovarin (THCV) and cannabidivarin (CBDV), in the treatment of different diseases and conditions has previously been described by the applicant in their International patent application WO 02/064109.

Specific ratios of THC and CBD or THCV and CBDV were reported to have been useful in the treatment or management of specific diseases or medical conditions.

Formulations containing specific, defined ratios of cannabinoids may be formulated from pure, synthetic cannabinoids or from extracts derived from the cannabis plant in combination with pharmaceutical carriers and excipients.

There is evidence that cannabinoids can act as an anti-inflammatory agent in vivo as is described by Gieringer (1996). The cannabinoids THC and CBD can be useful in the treatment of inflammatory diseases such as rheumatoid arthritis, as is described in the applicant's international patent application PCT/GB05/002233.

The cannabinoid CBD has also been shown to be an effective inhibitor of tumour cell migration. The applicants United Kingdom patent application GB0421900.2 describes the exposure of U87 human glioma cells to increasing concentrations of CBD. The migration of the tumour cells through a Boyden chamber was assessed. The IC₅₀ of CBD was determined to be 5.05±1.1 μM.

Surprisingly the applicants have found that administration of a medicament that contains a combination of the cannabinoids cannabidiol (CBD) and delta-9-tetrahydrocannabinol (THC) to patients with COPD results in a significant improvement in the patient's FEV₁/FVC ratios and an improvement in their anxiety and breathlessness as measured by visual analogue scores.

CBD is known not to act at CB₂ receptors and to act only very weakly at CB₁ receptors. Hence, the fact that such effects should be observed with combined administration of CBD and THC was particularly surprising given the prevailing view in the art that modulation of cannabinoid receptors (especially CB₂) is of key importance in the treatment of respiratory diseases such as COPD.

The improvements in anxiety observed following combined administration of CBD and THC were also surprising in view of the prevailing opinion in the art as regards the effects of cannabinoids (especially THC) on patient anxiety. There is much anecdotal evidence to link administration of THC with an increase in symptoms of anxiety. Zuardi et al. (Psychopharmacology (1982) 76: 245-250) describe a study into the action of CBD on the anxiety symptoms produced by Δ⁹-THC in normal subjects. The authors conclude that CBD may in part antigonize some symptoms of anxiety induced by administration of THC, suggesting that the two cannabinoids have independent and opposing effects. However, it has never previously been described or suggested that a combination of the two cannabinoids would be of any benefit in the treatment of anxiety associated with an underlying condition in human subjects.

### SUMMARY OF THE INVENTION

According to a first aspect of the present invention there is provided the use of a combination of cannabinoids cannabidiol (CBD) and delta-9-tetrahydrocannabinol (THC) in the manufacture of a pharmaceutical formulation for use in the treatment of Chronic Obstructive Pulmonary Disorder (COPD), wherein the ratio of CBD:THC by weight is between 10:1 and 1:10.

The invention also provides a method of treating Chronic Obstructive Pulmonary Disorder (COPD) in a human subject which comprises administering to a subject in need thereof a therapeutically effective amount of a combination of cannabinoids cannabidiol (CBD) and delta-9-tetrahydrocannabinol (THC), wherein the ratio of CBD:THC by weight is between 10:1 and 1:10.

According to a second aspect of the present invention there is provided the use of a combination of cannabinoids cannabidiol (CBD) and delta-9-tetrahydrocannabinol (THC) in the manufacture of a pharmaceutical formulation for use in the treatment of breathlessness, wherein the ratio of CBD:THC by weight is between 10:1 and 1:10.

The invention also provides a method of treating breathlessness in a human subject which comprises administering to a subject in need thereof a therapeutically effective amount of a combination of cannabinoids cannabidiol (CBD) and delta-9-tetrahydrocannabinol (THC), wherein the ratio of CBD:THC by weight is between 10:1 and 1:10.

According to a third aspect of the present invention there is provided the use of a combination of cannabinoids cannabidiol (CBD) and delta-9-tetrahydrocannabinol (THC) in the manufacture of a pharmaceutical formulation for use in the treatment of anxiety wherein the ratio of CBD:THC by weight is between 10:1 and 1:10.

The invention also provides a method of treating anxiety in a human subject which comprises administering to a subject in need thereof a therapeutically effective amount of a combination of cannabinoids cannabidiol (CBD) and delta-9-tetrahydrocannabinol (THC), wherein the ratio of CBD:THC by weight is between 10:1 and 1:10.

Preferred features of the invention will now be described in further detail. Features described as being preferred in relation to one aspect of the invention apply mutates mutandis to all other aspects, unless clearly stated otherwise.

Preferably the use of the cannabinoids in the manufacture of a pharmaceutical formulation are for use in the treatment of chronic bronchitis.

Preferably the use of the cannabinoids in the manufacture of a pharmaceutical formulation are for use in the treatment of chronic obstructive bronchitis.

Preferably the use of the cannabinoids in the manufacture of a pharmaceutical formulation are for use in the treatment of emphysema.

Preferably the use of the cannabinoids in the manufacture of a pharmaceutical formulation are for use in the treatment of breathlessness, wherein the breathlessness is caused by Chronic Obstructive Pulmonary Disorder (COPD).

Preferably the use of the cannabinoids in the manufacture of a pharmaceutical formulation are for use in the treatment of anxiety, wherein the anxiety is caused by Chronic Obstructive Pulmonary Disorder (COPD).

Preferably the ratio of CBD:THC by weight is between 5:1 and 1:5. More preferably the ratio of CBD:THC by weight is between 2:1 and 1:2. Most preferably the ratio of CBD:THC by weight is substantially 1:1.

Favourably the cannabinoids are packaged for delivery in a titratable dosage form.

Preferably the cannabinoid CBD is administered separately, simultaneously or sequentially to the cannabinoid THC.

The administration of a combination of cannabinoids such as THC and CBD to a patient could either be at the same time, wherein the cannabinoids would be contained in the same formulation. The cannabinoids could also be administered at separate times for example; a formulation containing CBD could be administered to a patient at a fixed time prior to a formulation containing THC in order to ameliorate some of the side effects of THC, which CBD is known to improve or vice versa. The two cannabinoids could also be administered consecutively to a patient if required.

The term “titrate” is defined as meaning that the patient is provided with a medication that is in such a form that smaller doses than the unit dose can be taken.

A “unit dose” is herein defined as a maximum dose of medication that can be taken at any one time or within a specified dosage period such as 3 hours.

Titration of doses are beneficial to the patient as they are able to take smaller of doses of the medication until the drug is efficacious. It is understandable that not all patients will require exactly the same dose of medication, for example patients of a larger build or faster metabolism may require a higher dose than that required by a patient that is of a smaller build. Different patients may also present with different degrees of complaints and as such may require larger or smaller doses in order to treat the complaint effectively. The benefits of such a dosage form over dosage forms such as tablets, where smaller doses are difficult to take, are therefore evident.

Unit dose ranges are preferably in the range of between 2 and 12 mg of each cannabinoid CBD and THC, more preferably in the range of 7 to 8.5 mg of each cannabinoid.

Preferably the maximum daily dosage dose of medicament is less than or equal to 120 mg CBD and less than or equal to 130 mg THC.

Preferably the cannabinoids are packaged for delivery such that delivery is targeted to an area selected from one or more of the following: sublingual, buccal, oral, rectal, nasal and the pulmonary system.

More preferably the cannabinoids are in the form selected from one or more of the following: gel, gel spray, tablet, liquid, capsule, for vaporisation and for nebulisation.

Additionally the pharmaceutical formulation further comprises one or more carrier solvents. Preferably the carrier solvents are ethanol and/or propylene glycol. More preferably the ratio of ethanol to propylene glycol is between 4:1 and 1:4. More preferably still the ratio is substantially 1:1.

Preferably the cannabinoids are present as a cannabis based medicine extract (CBME).

More preferably the combination of cannabinoids comprises:
a cannabis based medicinal extract which comprises THC at more than 90% of the total cannabinoid content in the extract; and
a cannabis based medicinal extract which comprises CBD at more than 90% of the total cannabinoid content in the extract.

Optionally the combination of cannabinoids are substantially pure.

Alternatively the combination of cannabinoids are synthetic.

In one embodiment the CBME are produced by extraction with supercritical or subcritical CO₂. In an alternative embodiment the CBME are produced by extraction from plant material by volatilisation with a heated gas. Preferably the CBME contain all of the naturally occurring cannabinoids in the plant material. Alternatively synthetic or highly purified isolates of the cannabinoids can be used.

The combination of cannabinoids may be administered in combination with one or more other drugs.

More preferably the combination of cannabinoids are administered in addition to one or more bronchodilatory drugs.

Examples of bronchodilatory drugs include but are not limited to albuterol, aminophylline, bitolterol, ephedrine, epinephrine, fenoterol, isoetharine, isoproterenol, metaproterenol, oxtriphylline, pirbuterol, procaterol salmeterol, terbutaline, and theophylline.

More preferably still the combination of cannabinoids are administered in addition to one or more anti-inflammatory drugs.

Examples of anti-inflammatory drugs include but are not limited to diclofenac, diflunisal, etodolac, fenoprofen, floctafenine, flurbiprofen, ibuprofen, ketoproen, meclofenamate, mefanamic acid, meloxicam, nabumetone, naproxen, oxaprozin, phenylbutazone, piroxicam, sulindac, tenoxicam, tiaprofenic acid and tolmetin.

Preferably the combination of cannabinoids are administered in addition to one or more mucolytic drugs.

Examples of mucolytic drugs include but are not limited to acetylcysteine, carbocisteine, dornase alfa, methyl cysteine and stepronin.

Preferably the combination of cannabinoids are administered in addition to one or more antibiotic drugs.

Examples of the different groups of antibiotic drugs that may be used include but are not limited to aminoglycosides, antimycobacterials, cephalosporins and related beta lactams, chloramphenicols, glycopeptides, lincosamides, macrolides, penicillins, quinolones, sulphonamides, diaminopyramidines and tetracyclines.

The term “in combination” refers to administration of the cannabinoids at the same time and in the same formulation as the additional drug.

The term “in addition to” refers to administration of the cannabinoids to patient who is already being administered additional drugs.

More preferably the combination of cannabinoids are administered separately, simultaneously or sequentially to the one or more other drugs.

The term “approximately equal” is used to refer to ratios of cannabinoids which are in the range of between 0.9:1 to 1:0.9 (THC:CBD). Additionally the term “1:1” is taken herein to refer to approximately equal amounts of cannabinoids.

### BRIEF DESCRIPTION OF THE DRAWINGS

Certain aspects of this invention are further described, by way of example only, with reference to the accompanying drawings in which:

FIG. 1 shows the FEV₁/FVC ratio of all patients and subgroups; and

FIG. 2 shows the Visual Analogue Scale scores of all patients for breathlessness and anxiety.

### SPECIFIC DESCRIPTION

A cannabis based medicine extract (CBME) was prepared as outlined in Example 1 and contained approximately equal amounts of the cannabinoids THC and CBD and this was administered to patients with chronic obstructive pulmonary disease (COPD) in order to assess the effects of the cannabinoids on these patients symptoms.

### Example 1

### Preparation of Cannabis Based Medicine Extracts (CBME)

Medicinal cannabis was produced and prepared with reference to the method disclosed in WO 02/064109 (Example 15). The resulting plant material was processed as described in the flow chart below. The process of manufacture of a High THC or High CBD cannabis based medicine extract is described.

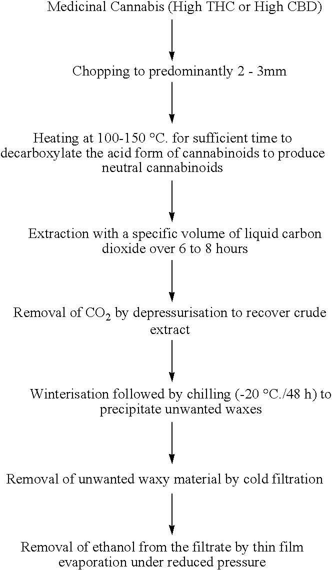

The resulting extract is referred to as a cannabis based medicinal drug extract and is also classified as a Botanical Drug Substance according to the US Food and Drug Administration Guidance for Industry Botanical Drug Products.

The quantity of cannabinoid in the CBME can be accurately assessed by way of measurement by HPLC with reference to the method disclosed in WO 02/064109 (Example 16), the contents of which are incorporated herein in their entirety by reference.

### Example 2

### Dose-Escalation and Safety Study of a Cannabis-Based Medicine in Patients with Moderate to Severe Chronic Obstructive Pulmonary Disease (COPD)

In a dose finding study a single dose of the test article was administered to 15 hospitalised patients who had recovered from an acute COPD exacerbation. The test article that was studied was CBME THC:CBD (1:1) in ethanol:propylene glycol (1:1). Patients were administered with the test article once in the morning. The test article was delivered as a sublingual actuation, with each actuation providing a dose of 2.7 mg THC and 2.5 mg CBD.

The study population were male or female patients with a mean age of 72 (8.6 SD) with a range of 46-82, who have moderate to severe COPD with a mean FEV₁ (forced expiratory volume over 1 second) of 0.71 (SD 0.30) and 33% of predicted. The study population had a mean MRC breathlessness score of 4.2 and an ECOG performance score of 2.3.

Measurements were taken from each patient at set times: 24 hours prior to dosing, at baseline and 2, 4, 6 and 24 hours post dosing.

These measurements were:

1. Spirometry—this measures the patients:
FEV₁ (Forced Expiratory Volume in one second), which is the amount of air that can be blown out of the lungs within one second. In a patient with normal lungs and airways the patient can usually blow most of the air from their lungs in one second.
FVC (Forced Vital Capacity), which is the total amount of air that can be blown out of the lungs.
FEV₁/FVC, this is the proportion of air in the patient's lung that can be blown out in one second.
   2. Visual Analogue Scores (VAS scale 0-10 cm) for anxiety and breathlessness; and
   3. Blood pressure.

Arterial blood gas measurements were taken at baseline, 4 and 24 hours post dosing.

Oxygen saturation, 4% oxygen dip rate per hour, Oxygen saturation time spent below 90% and heart rate were recorded continuously for the period 24 hours prior to dosing to 24 hours post dosing.

The mean differences of variables at any time against baseline were compared using paired samples t-test (SPSS 12.0.2).

Surprisingly the cannabis based medicine extract containing approximately equal quantities of THC and CBD was shown to produce a significant improvement in the FEV₁/FVC ratios at 2, 4 and 6 hours post dosing.

The test article contained delta-9-tetrahydrocannabinol (THC) at a concentration of 27 mg/ml and cannabidiol (CBD) at a concentration of 25 mg/ml in ethanol:propylene glycol (50:50) excipient. The CBME was presented in a pump action spray where each activation delivers 100 μl of spray, containing THC (2.7 mg) and CBD (2.5 mg). Each actuation was delivered sublingually to the patient.

The subjects in the study were numbered sequentially and patients 1 to 5 received two sublingual actuations (5.4 mg THC and 5.0 mg CBD), patients 6 to 10 received three actuations each (8.1 mg THC and 7.5 mg CBD) and patients 11 to 15 received four actuations of the test article each (10.8 mg THC and 10.0 mg CBD).

### Results:

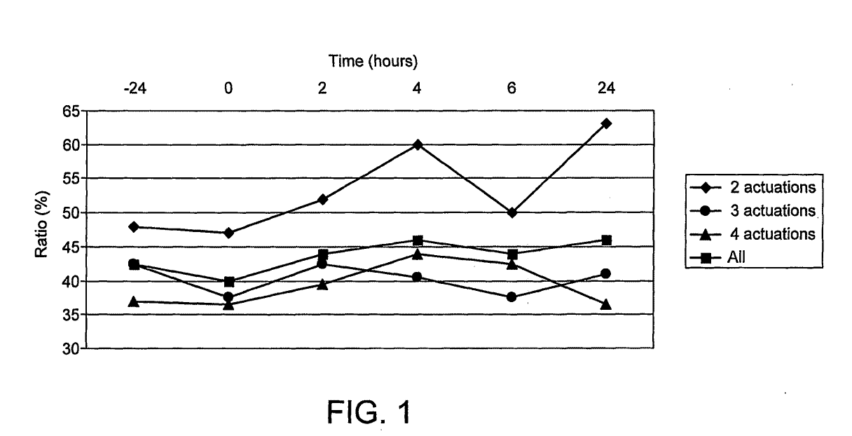

FIG. 1 shows that there was a significant improvement in the FEV₁/FVC ratios at 2, 4 and 6 hours post dosing. There were no changes in oxygen saturation, 4% oxygen dip rate per hour, oxygen saturation time spent below 90%, blood gases and mean heart rate.

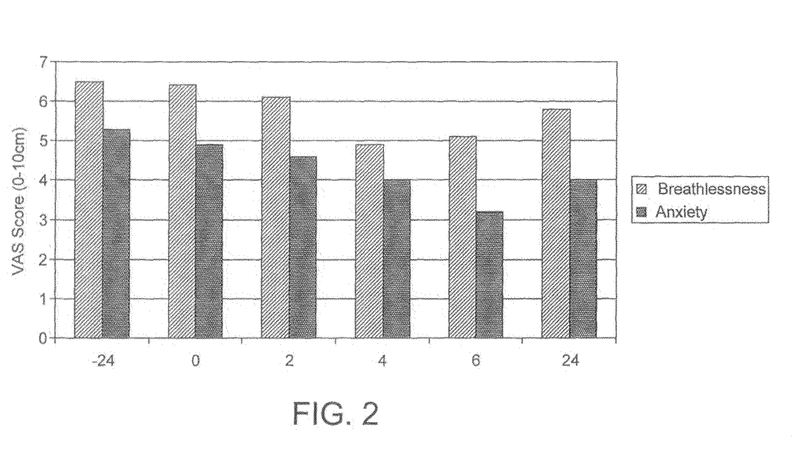

FIG. 2 shows that the mean anxiety and breathlessness scores in all patients showed a trend of improvement. This was statistically significant for breathlessness in patients who received three actuations at 2 hours post dosing versus 4 hours post dosing (p=0.034) and versus 6 hours post dosing (p=0.038).

Some patients who received 4 actuations of the study medication recorded a worsening of their breathlessness symptoms (increase of 0.7 points) at 6 hours post dosing.

Seven of the patients gave positive comments such as feeling stronger, relaxed and increased appetite.

It can therefore be concluded that a medication that contains approximately equal amounts of THC and CBD offers a new treatment option in the treatment of patients with COPD. It is useful also to note that improvement of symptoms of breathlessness and anxiety along with the significant improvement in the FEV₁/FVC ratios there was no deterioration of vital parameters such as oxygenation.

1.-27. (canceled)

28. A method of treating Chronic Obstructive Pulmonary Disorder (COPD) in a human subject which comprises administering to a subject in need thereof a therapeutically effective amount of a combination of cannabinoids cannabidiol (CBD) and delta-9-tetrahydrocannabinol (THC), wherein the ratio of CBD:THC by weight is between 10:1 and 1:10.

29. A method of treating breathlessness in a human subject which comprises administering to a subject in need thereof a therapeutically effective amount of a combination of cannabinoids cannabidiol (CBD) and delta-9-tetrahydrocannabinol (THC), wherein the ratio of CBD:THC by weight is between 10:1 and 1:10.

30. A method of treating anxiety in a human subject which comprises administering to a subject in need thereof a therapeutically effective amount of a combination of cannabinoids cannabidiol (CBD) and delta-9-tetrahydrocannabinol (THC), wherein the ratio of CBD:THC by weight is between 10:1 and 1:10.

31. (canceled)

32. The method as claimed in claim 28, wherein the COPD is chronic bronchitis, chronic obstructive bronchitis or emphysema.

33. The method as claimed claim 28, wherein the ratio of CBD:THC by weight is between 5:1 and 1:5.

34. The method as claimed claim 28, wherein the ratio of CBD:THC by weight is between 2:1 and 1:2.

35. The method as claimed claim 28, wherein the ratio of CBD:THC by weight is substantially 1:1.

36. The method as claimed claim 28, wherein the cannabinoids are packaged for delivery in a titratable dosage form.

37. The method as claimed in claim 28, wherein the cannabinoid CBD is administered separately, simultaneously or sequentially to the cannabinoid THC.

38. The method as claimed in claim 28, wherein a unit dose taken by a patient is in the range of 2-12 mg of each cannabinoid.

39. The method as claimed in claim 38, wherein a unit dose taken by a patient is in the range of 7-8.5 mg of each cannabinoid.

40. The method as claimed in claim 28, wherein the maximum daily dosage dose of each cannabinoid is less than or equal to 120 mg of CBD and less than or equal to 130 mg of THC.

41. The method as claimed in claim 28, wherein the cannabinoids are packaged for delivery such that delivery is targeted to an area selected from the group consisting of: sublingual, buccal, oral, rectal, nasal and the pulmonary system.

42. The method as claimed in claim 41, wherein the cannabinoids are in a form selected from the group consisting of: gel, gel spray, tablet, liquid, capsule, for vaporisation and for nebulisation.

43. The method as claimed in claim 28, wherein the cannabinoids are present as a cannabis based medicine extract (CBME).

44. The method as claimed in claim 28, wherein the combination of cannabinoids comprises:
a) a cannabis based medicinal extract which comprises THC at more than 90% of the total cannabinoid content in the extract; and
b) a cannabis based medicinal extract which comprises CBD at more than 90% of the total cannabinoid content in the extract.

45. The method as claimed claim 28, wherein the cannabinoids are substantially pure.

46. The method as claimed in claim 28, wherein the cannabinoids are synthetic.

47. The method as claimed in claim 28, wherein the cannabinoids are administered in combination with one or more other drugs.

48. The method as claimed in claim 47, wherein the cannabinoids are administered in addition to one or more bronchodilatory drugs, one or more anti-inflammatory drugs, one or more mucolytic drugs and/or one or more antibiotic drugs.

49. The method as claimed in claim 29, wherein the breathlessness is caused by Chronic Obstructive Pulmonary Disorder (COPD).

50. The method as claimed claim 29, wherein the ratio of CBD:THC by weight is between 5:1 and 1:5.

51. The method as claimed claim 29, wherein the ratio of CBD:THC by weight is between 2:1 and 1:2.

52. The method as claimed claim 29, wherein the ratio of CBD:THC by weight is substantially 1:1.

53. The method as claimed claim 29, wherein the cannabinoids are packaged for delivery in a titratable dosage form.

54. The method as claimed in claim 29, wherein the cannabinoid CBD is administered separately, simultaneously or sequentially to the cannabinoid THC.

55. The method as claimed in claim 29, wherein a unit dose taken by a patient is in the range of 2-12 mg of each cannabinoid.

56. The method as claimed in claim 55, wherein a unit dose taken by a patient is in the range of 7-8.5 mg of each cannabinoid.

57. The method as claimed in claim 29, wherein the maximum daily dosage dose of each cannabinoid is less than or equal to 120 mg of CBD and less than or equal to 130 mg of THC.

58. The method as claimed in claim 29, wherein the cannabinoids are packaged for delivery such that delivery is targeted to an area selected from the group consisting of: sublingual, buccal, oral, rectal, nasal and the pulmonary system.

59. The method as claimed in claim 58, wherein the cannabinoids are in a form selected from the group consisting of: gel, gel spray, tablet, liquid, capsule, for vaporisation and for nebulisation.

60. The method as claimed in claim 29, wherein the cannabinoids are present as a cannabis based medicine extract (CBME).

61. The method as claimed in claim 29, wherein the combination of cannabinoids comprises:
a) a cannabis based medicinal extract which comprises THC at more than 90% of the total cannabinoid content in the extract; and
b) a cannabis based medicinal extract which comprises CBD at more than 90% of the total cannabinoid content in the extract.

62. The method as claimed claim 29, wherein the cannabinoids are substantially pure.

63. The method as claimed in claim 29, wherein the cannabinoids are synthetic.

64. The method as claimed in claim 29, wherein the cannabinoids are administered in combination with one or more other drugs.

65. The method as claimed in claim 64, wherein the cannabinoids are administered in addition to one or more bronchodilatory drugs, one or more anti-inflammatory drugs, one or more mucolytic drugs and/or one or more antibiotic drugs.

66. The method as claimed in claim 30, wherein the anxiety is caused by Chronic Obstructive Pulmonary Disorder (COPD).

67. The method as claimed claim 30, wherein the ratio of CBD:THC by weight is between 5:1 and 1:5.

68. The method as claimed claim 30, wherein the ratio of CBD:THC by weight is between 2:1 and 1:2.

69. The method as claimed claim 30, wherein the ratio of CBD:THC by weight is substantially 1:1.

70. The method as claimed claim 30, wherein the cannabinoids are packaged for delivery in a titratable dosage form.

71. The method as claimed in claim 30, wherein the cannabinoid CBD is administered separately, simultaneously or sequentially to the cannabinoid THC.

72. The method as claimed in claim 30, wherein a unit dose taken by a patient is in the range of 2-12 mg of each cannabinoid.

73. The method as claimed in claim 72, wherein a unit dose taken by a patient is in the range of 7-8.5 mg of each cannabinoid.

74. The method as claimed in claim 30, wherein the maximum daily dosage dose of each cannabinoid is less than or equal to 120 mg of CBD and less than or equal to 130 mg of THC.

75. The method as claimed in claim 30, wherein the cannabinoids are packaged for delivery such that delivery is targeted to an area selected from the group consisting of: sublingual, buccal, oral, rectal, nasal and the pulmonary system.

76. The method as claimed in claim 75, wherein the cannabinoids are in a form selected from the group consisting of: gel, gel spray, tablet, liquid, capsule, for vaporisation and for nebulisation.

77. The method as claimed in claim 30, wherein the cannabinoids are present as a cannabis based medicine extract (CBME).

78. The method as claimed in claim 30, wherein the combination of cannabinoids comprises:
a) a cannabis based medicinal extract which comprises THC at more than 90% of the total cannabinoid content in the extract; and
b) a cannabis based medicinal extract which comprises CBD at more than 90% of the total cannabinoid content in the extract.

79. The method as claimed claim 30, wherein the cannabinoids are substantially pure.

80. The method as claimed in claim 30, wherein the cannabinoids are synthetic.

81. The method as claimed in claim 30, wherein the cannabinoids are administered in combination with one or more other drugs.

82. The method as claimed in claim 81, wherein the cannabinoids are administered in addition to one or more bronchodilatory drugs, one or more anti-inflammatory drugs, one or more mucolytic drugs and/or one or more antibiotic drugs.
